(19) United States
(12) Patent Application Publication
(10) Pub. No.: US 2021/0065892 A1
(43) Pub. Date: Mar. 4, 2021

(54) OUT-PATIENT HEALTH MANAGEMENT SYSTEM AND A METHOD TO OPERATE THE SAME

(71) Applicant: Rajesh Vangara, Concord, NC (US)

(72) Inventors: Vipin Vangara, Charlotte, NC (US); Ravi Chandar Vangara, Bangalore (IN); Siddharth Vangara, Concord, NC (US)

(21) Appl. No.: 16/555,480

(22) Filed: Aug. 29, 2019

(51) Int. Cl. G16H 40/67; G16H 50/30; G16H 20/10; G16H 10/60

(52) U.S. Cl. CPC G16H 40/67; G16H 10/60; G16H 20/10; G16H 50/30

(57) ABSTRACT

A method of health monitoring for out-patient is disclosed. The method includes receiving health vital data from the one or more external user devices, organizing received health vital data to create a medical data file representative of medical history of a user. The method includes analysing the processed medical data file to generate one or more patterns corresponding to the health vital data and generating one or more recommendations provided by a healthcare provider based on the one or more patterns. The method includes generating alerts to health care practitioner, kith & kin, health care practitioner based on out-patient's permissions. The method includes generating a benchmark result based on the one or more patterns and the one or more recommendations by the healthcare provider. The method includes recommending the benchmark result of the user to one or more patients comprising same race, age, gender and weight that of the user.

### BACKGROUND

Embodiments of a present disclosure relate to Out-patient healthcare management and more particularly an out-patient healthcare management system and a method to operate the same.

The healthcare system includes a variety of participants, including doctors, hospitals, insurance carriers, and patients. Such participants frequently rely on each other for the information necessary to perform their respective roles because individual care is delivered and paid for in numerous locations by individuals and organizations that are typically unrelated. As a result, an excess of healthcare information storage and retrieval systems are required to support the heavy flow of information between such participants related to patient care. However, critical patient data is stored across many different locations using legacy mainframe and client-server systems that may be incompatible and may store information in non-standardized formats.

Furthermore, to ensure proper patient diagnosis and treatment, health care providers must often request patient information by cellular phone or fax from hospitals, laboratories or other providers. However, disparate systems and information delivery procedures maintained by a number of independent healthcare system constituents lead to gaps in timely delivery of critical information and compromise the overall quality of medical care.

Moreover, a typical healthcare practice is concentrated within a given specialty, an average patient may be using services of a number of different specialists, each potentially having only a partial view of the patient's medical status such as healthcare providers do not have quantitative and qualitative data about the out-patient's adoption. Also, patients do not know how the other patients of similar age, gender, race and weight are coping with the chronic diseases on similar treatment. Therefore, potential gaps in complete medical records reduce the value of medical advice given to the patient by each healthcare provider. Also, typical healthcare practices don not provide a global platform which captures all the vitals and transfers to endocrinologist or diabetologist at regular intervals or as opted or recommended.

Currently existing solutions utilize centralized storage system of healthcare information. However, such centralized storage system has failed to incorporate real-time analysis of a patient's healthcare information in order to expeditiously identify potential medical issues which may require attention.

Hence, there is a need for an improved health monitoring system for out-patient to address the aforementioned issue(s).

### BRIEF DESCRIPTION

In accordance with an embodiment of the present disclosure, a health monitoring system for out-patient is provided. The system includes a centralized platform located on a server and operatively coupled to one or more external user devices. The centralized platform includes a data receiving subsystem configured to receive health vital data from the one or more external user devices. The centralized platform also includes a data processing subsystem operatively coupled to the data receiving subsystem. The data processing subsystem is configured to organize received health vital data to create a medical data file representative of medical history of a user. The centralized platform further includes a medical data analysis subsystem operatively coupled to the data processing subsystem. The medical data analysis subsystem is configured to analyse the medical data file using one or more analysis techniques. The medical data analysis subsystem is also configured to generate one or more patterns corresponding to the health vital data based on analysed results. The centralized platform further includes a recommendation subsystem operatively coupled to the analysis subsystem. The recommendation subsystem is configured to generate a report with one or more recommendations, wherein the one or more recommendations are provided by a healthcare provider based on the one or more patterns. The centralized platform further includes a benchmark subsystem operatively coupled to the recommendation subsystem and medical data analysis subsystem. The benchmark subsystem is configured to generate a benchmark result based on the one or more patterns and the report with the one or more recommendations by the healthcare provider. The benchmark subsystem is also configured to provide the benchmark result of the user to one or more patients comprising at least one of a race, an age, a gender and weight corresponding to at least one of a race, an age, a gender and weight of the user based on permission of the user.

In accordance with another embodiment of the present disclosure, a method of health monitoring for out-patient is provided. The method includes receiving, by a data receiving subsystem, health vital data from the one or more external user devices. The method also includes organizing, by a data processing subsystem, received health vital data to create a medical data file representative of medical history of a user. The method further includes analysing, by a medical data analysis subsystem, the medical data file using one or more analysis techniques. The method further includes generating, by the medical data analysis subsystem, one or more patterns corresponding to the health vital data based on analysed results. The method further generating, by a recommendation subsystem, a report with one or more recommendations, wherein the one or more recommendations are provided by a healthcare provider based on the one or more patterns. The method further includes generating, by a benchmark subsystem, a benchmark result based on the one or more patterns and e a report with the one or more recommendations by the healthcare provider. The method further includes providing, by the benchmark subsystem, the benchmark result of the user to one or more patients comprising at least one of a race, an age, a gender and weight corresponding to at least one of a race, an age, a gender and weight of the user.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a health monitoring system for an out-patient in accordance with an embodiment of the present disclosure;

FIG. 2 is a block diagram representation of an exemplary embodiment of the health monitoring system for an out-patient of FIG. 1 in accordance with an embodiment of the present disclosure; and

FIG. 3 is a block diagram of a computer or a server in accordance with an embodiment of the present disclosure; and

FIG. 4 is a flow chart representing the steps involved in a method of health monitoring for an out-patient in accordance with the embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a health monitoring system for an out-patient. The system includes a centralized platform located on a server and operatively coupled to one or more external user devices. The centralized platform includes a data receiving subsystem configured to receive health vital data from the one or more external user devices. The centralized platform also includes a data processing subsystem operatively coupled to the data receiving subsystem. The data processing subsystem is configured to organize received health vital data to create a medical data file representative of medical history of a user. The centralized platform further includes a medical data analysis subsystem operatively coupled to the data processing subsystem. The medical data analysis subsystem is configured to analyse the medical data file using one or more analysis techniques. The medical data analysis subsystem is also configured to generate one or more patterns corresponding to the health vital data based on analysed results. The centralized platform further includes a recommendation subsystem operatively coupled to the analysis subsystem. The recommendation subsystem is configured to generate a report with one or more recommendations, wherein the one or more recommendations are provided by a healthcare provider based on the one or more patterns. The centralized platform further includes a benchmark subsystem operatively coupled to the recommendation subsystem and medical data analysis subsystem. The benchmark subsystem is configured to generate a benchmark result based on the one or more patterns and a report with the one or more recommendations by the healthcare provider. The benchmark subsystem is also configured to provide the benchmark result of the user to one or more patients comprising at least one of a race, an age, a gender and weight corresponding to at least one of a race, an age, a gender and weight of the user based on permission of the user.

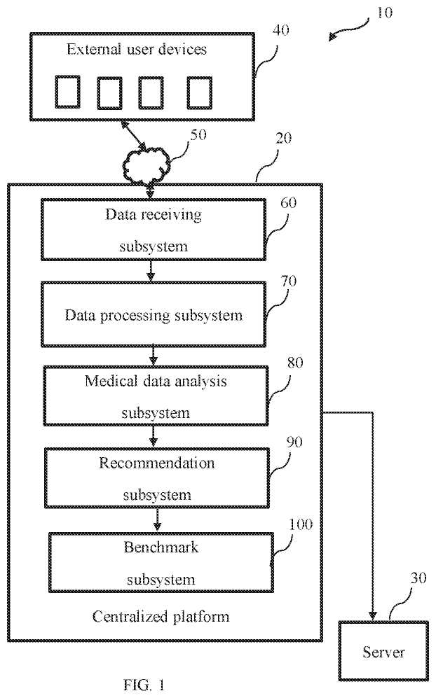

FIG. 1 is a block diagram representation of a health monitoring system 10 for an out-patient in accordance with an embodiment of the present disclosure. The system 10 includes a centralized platform 20 located on a server 30 and operatively coupled to one or more external user devices 40. In one embodiment, the server 30 may include an on premises computing platform server or a private server. In some embodiments, the one or more external user devices 40 may include at least one of a weighing scale, a wearable device includes a pedometer, a pulse rate meter, sleep monitoring device, a food scale, an image acquisition device, a blood pressure meter, a gluco meter, an insulin injector, one or more cardiac tools and the like. In a specific embodiment, the one or more external user device 40 is communicatively coupled to the centralized platform 20 via a communication network 50. In such embodiment, the communication network 50 may include a wired communication medium such as local area network (LAN). In another embodiment, the communication network 50 may include a wireless communication medium such as radio frequency (RF) communication, Bluetooth, Infrared (IR) communication, wireless fidelity (wi-fi) or the like.

The centralized platform 20 includes a data receiving subsystem 60 configured to receive health vital data from the one or more external user devices 40. In one embodiment, the health vital data may include at least one of bodyweight, blood pressure, glucose level, cholesterol level, pulse rate, amount of sleep, steps count, exercise-related data, weight and calorie intake of food, electrocardiogram data, haemoglobin level, oxygen level in blood insulin, Nicotine level through Saliva and the like. In some embodiments, the data receiving subsystem 60 may be configured to receive information corresponding to oral or injectable medication of the user via an image acquisition device. In such embodiment, the image acquisition may include a video camera, a still camera and the like.

Furthermore, the centralized platform 20 includes a data processing subsystem 70 operatively coupled to the data receiving subsystem 60. The data processing subsystem 70 is configured to organize received health vital data to create a medical data file representative of medical history of a user. In one embodiment, the medical history may include blood pressure (BP), glucose level, cholesterol level, pulse rate, electrocardiogram (ECG), haemoglobin level, oxygen level in blood, insulin level, Nicotine level through saliva and the like. The data processing subsystem 70 is also configured to process the medical data file.

Moreover, the centralized platform 20 further includes a medical data analysis subsystem 80 operatively coupled to the data processing subsystem 70. The medical data analysis subsystem 80 is configured to analyse the processed medical data file using one or more analysis techniques. In one embodiment, the one or more analysis technique may include at least one of a descriptive analysis technique, a predictive analysis technique, a prescriptive analysis technique, a comparative analysis technique and the like. The medical data analysis subsystem 80 is also configured to generate one or more patterns corresponding to the health vital data based on analysed results. For example, the one or more patterns may include number of patients in critical condition that need immediate attention by the endocrinologist, Patients that are improving in their health condition and patients that are neither improving and nor deteriorating. Based on the one or more patterns, the health care providers may be able to identify the health condition of the patient at regular intervals (weekly, Fortnight, Monthly data).

In one embodiment, the medical data analysis subsystem 80 may be configured to receive an access request from a third-party user to access the health vital data of the user. In one embodiment, the access may be provided digitally. In such embodiment, the medical data analysis subsystem 80 may also be configured to generate a permission grant request to access the health vital data by the third-party user and provide the access to transmit or allow, to access the data to the third-party user like immediate or designated family members, dietician, payer (health insurance companies) and Pharma company.

The centralized platform 20 further includes a recommendation subsystem 90 which is operatively coupled to the medical data analysis subsystem 80. The recommendation subsystem 90 is configured to enable a healthcare provider to monitor the one or more patterns. The recommendation subsystem 90 is also configured to generate a report with one or more recommendations, wherein the one or more recommendations are provided by a healthcare provider based on the one or more patterns. In some embodiments, the recommendation subsystem 90 may be configured to suggest one or more tools to the user corresponding to the one or more recommendations based on the one or more patterns. In one embodiment, the recommendation subsystem 90 enables the healthcare provider to provide a digital prescription along with the drug administering dosage and time to administer the patient after visiting the healthcare provider.

Subsequently, the centralized platform 20 further includes a benchmark subsystem 100 operatively coupled to the recommendation subsystem 90 and the medical data analysis subsystem 80. The benchmark subsystem 100 is configured to generate a benchmark result based on the one or more patterns and the report with the one or more recommendations by the healthcare provider. The one or more recommendations are not displayed to the patient and used for obtaining better benchmarking results. Here, the category corresponds to the one or more patterns obtained from the health vital data. The benchmark subsystem 100 is also configured to provide the benchmark result of the user to one or more patients including at least one of a race, an age, a gender and weight corresponding to at least one of a race, an age, a gender and weight of the user. In one embodiment, the benchmark results may be provided to the patient based on the permission of the user.

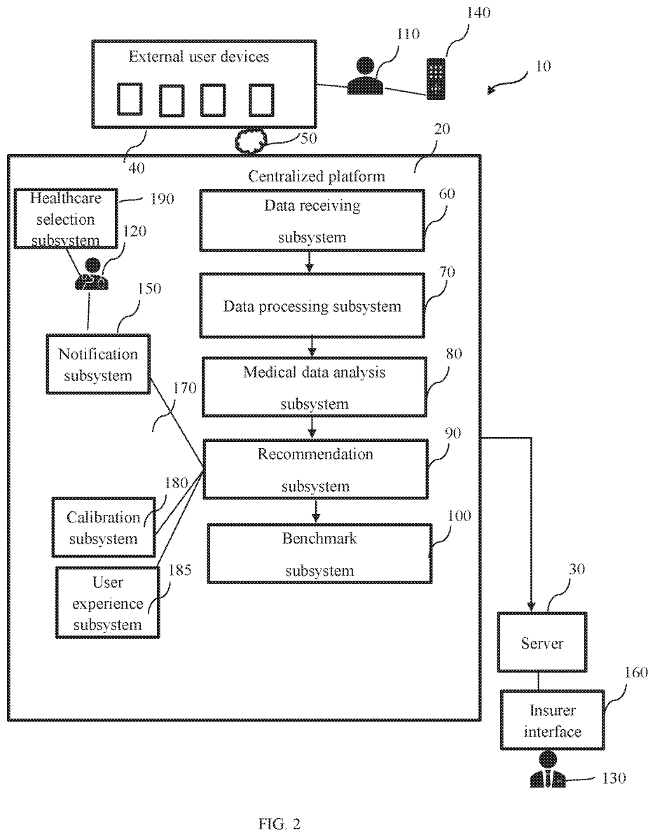

FIG. 2 is a block diagram representation of one embodiment of the health monitoring system 10 for an out-patient of FIG. 2 in accordance with an embodiment of the present disclosure. Considering an example where a user is a diabetic patient and a healthcare provider is a diabetologist. The diabetic patient 110 is using the health monitoring system 10 which includes the centralized platform 20. The centralized platform 20 integrating the one or more external user devices 30 that are used by the diabetic patient for management with regular feedback mechanism of maintenance to healthcare provider such as diabetologist 120 and insurers 130, through which the diabetologist 120 may be able to monitor diabetic patient's diet, exercise and stress at regular intervals as prescribed. The centralized platform 20 includes the data receiving subsystem 60 which receives health vital data (bodyweight of the diabetic patient, glucose level, exercise-related data, Sleep data, calorie of food, insulin, nicotine and the like) from the one or more external user devices 40 such as weighing scale, a food scale, a gluco meter, an insulin injector, Blood Pressure Meter and pulse oximeter. The data receiving subsystem 60 also receives images of medicines being consumed by the diabetic patient at intervals via smart phone camera 140.

Further, the data processing subsystem 70 of the centralized platform 20 organizes received health vital data to create a medical data file representative of medical history of the diabetic patient 110. The data processing subsystem 70 further process the medical data file and transmit the processed medical data file to the medical data analysis subsystem 80 of the centralized platform 20. The medical data analysis subsystem 80 analysis the processed medical data file using one or more analysis techniques. The medical data analysis subsystem 80 is configured to generate one or more patterns such as a pattern with gradual increase in the glucose level of the diabetic patient 110 which may be a result of a plurality of factors such as high intake of carbohydrates, high stress or lack of exercise. Based on the patterns, the recommendation subsystem 90 is configured to generate a report, wherein the report includes the recommendations provided by the diabetologist 120. In such case, based on the glucose level of the diabetic patient 110 the diabetologist 120 may suggest exercises or may give suitable suggestions, wherein such suggestions are presented to the diabetic patient 110 in the form of report.

In some cases, the benchmark subsystem 100 of the centralized subsystem 20 may be configured to analyse the health vital data and the recommendations provided by the diabetologist 120 of the diabetic patient 110. Based on the analysed results, the benchmark subsystem 100 may be configured to generate a benchmark result for a user with same age, gender, weight that of the patient 110. The benchmark result is displayed to the user based on the permission of the patient. The recommendations are utilized to improve the benchmark result and hence, the health care provider's unique medication recommendations are not shared to the other patients.

In one embodiment, the centralized platform 20 may include a notification subsystem 150 which is coupled to the recommendation subsystem 80. The notification subsystem 80 is configured to receive one or more reminders from the healthcare provider 120 for the medicine dosages and preventive steps. In another embodiment, the notification subsystem 150 may be configured to generate one or more alerts for the user's 110 kith and kin wherein the one or more alerts are corresponding to monitored health vital data and one or more patterns. Similarly, the healthcare provider receives weekly, monthly, fortnight patterns of the patient to judge the progress of the patient and practices adopted by the patient based on the healthcare provider's recommendations. In case of any adverse conditions of patient, the healthcare provider may receive the one or more alerts corresponding to health condition of the patient on an interface associated with the healthcare provider. In some embodiments, the system 10 may include an insurer interface 160 operatively coupled to the centralized platform 20. The insurer interface 160 is configured to receive a digital prescription from the healthcare provider 120. The digital prescription is prescribed to an insured user. In one embodiment, the insurer interface 160 may be configured to receive a health progress report of the insured user and the one or more tools used by the insured user. In such embodiment, the health progress report may include at least one of medicine consumption, diet pattern, exercise data and sleep pattern, weight and diet management trends.

In some embodiments, the centralized platform 20 may include a calibration subsystem 180 operatively coupled to the recommendation subsystem 90. The calibration subsystem 180 is configured to enable the user 110 to remotely calibrate at least one of the one or more external user devices 40 and the one or tools used by the user 110.

In a specific embodiment, the centralized platform 20 may include a user experience sharing subsystem 185 operatively coupled to rating subsystem 170. The user experience sharing subsystem 185 is configured to receive a user experience corresponding to at least one of daily routine of diet, calories intake, exercise data, sleep patterns via a textual means. In a preferred embodiment, the centralized platform 20 may include a healthcare selection subsystem 190 which is configured to enable the user to select a healthcare provider based on a location of the user 110. With the continued reference to above mentioned example, when the diabetic patient 110 is travelling abroad then, using the healthcare selection subsystem 190 the diabetic patient 110 may select the diabetologist 130 of his choice and may share the health vital data. Based on the shared health vital data, the selected diabetologist may provide one or more recommendations to the diabetic patient 110 as per his health condition at that time.

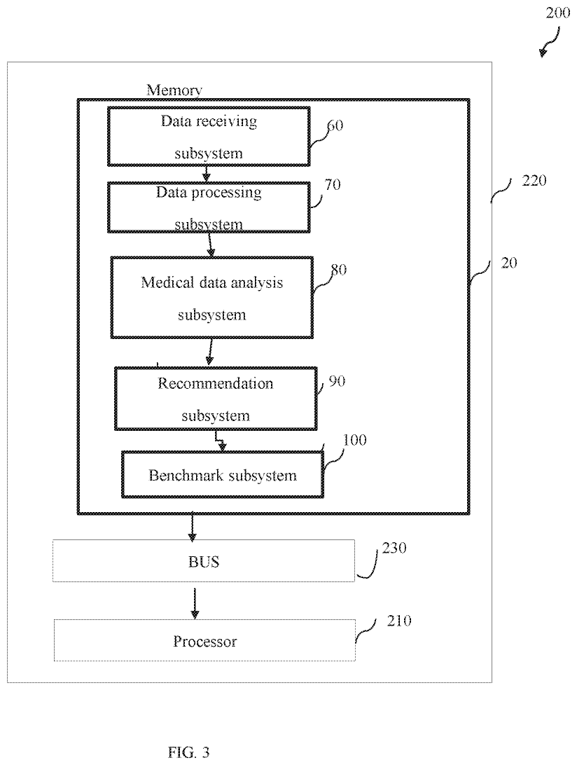

FIG. 3 is a block diagram of a computer or a server 200 in accordance with an embodiment of the present disclosure. The server includes processor(s) 210, and memory 220 operatively coupled to the bus 230. The processor 210 and memory 220 are substantially to similar to the centralized platform 20 of FIG. 1.

The processor(s) 210, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 220 includes a plurality of subsystems stored in the form of executable program which instructs the processor 210 to perform the method steps illustrated in FIG. 1. The memory 220 is substantially similar to the system 10 of FIG. 1. The memory 220 has following subsystems: a data receiving subsystem 60, a data processing subsystem 70, a medical data analysis subsystem 80, a recommendation subsystem 90 and a benchmark subsystem 100.

The data receiving subsystem 60 configured to receive health vital data from the one or more external user devices. The data processing subsystem 70 operatively coupled to the data receiving subsystem 60. The data processing subsystem 70 is configured to organize received health vital data to create a medical data file representative of medical history of a user. The medical data analysis subsystem 80 operatively coupled to the data processing subsystem 70. The medical data analysis subsystem 80 is configured to analyse the processed medical data file using one or more analysis techniques. The medical data analysis subsystem 80 is also configured to generate one or more patterns corresponding to the health vital data based on analysed results.

The recommendation subsystem 90 operatively coupled to the medical data analysis subsystem 80. The recommendation subsystem 90 is configured to generate a report with one or more recommendations, wherein the recommendations are provided by a healthcare provider based on the one or more patterns. The benchmark subsystem 100 operatively coupled to the recommendation subsystem 90 and medical data analysis subsystem 80. The benchmark subsystem 100 is configured to generate a benchmark result based on the one or more patterns and the report with the one or more recommendations by the healthcare provider. The benchmark subsystem 100 is also configured to provide the benchmark result of the user to one or more patients comprising at least one of a race, an age, a gender and weight corresponding to at least one of a race, an age, a gender and weight of the user based on the permission of the user.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the processor(s) 210.

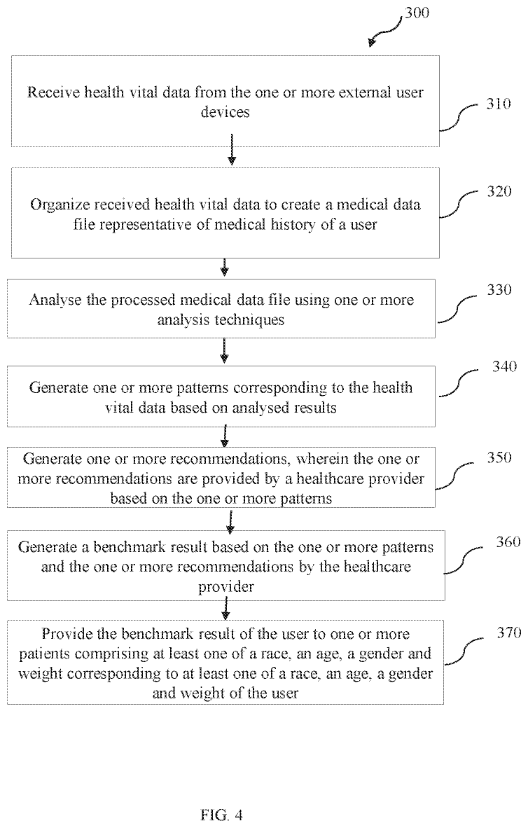

FIG. 4 is a flow chart representing the steps involved in a method 300 of health monitoring for an out-patient in accordance with an embodiment of the present disclosure. The method 300 includes receiving, by a data receiving subsystem, health vital data from the one or more external user devices in step 310. In one embodiment, receiving the health vital data from the one or more external user devices may include receiving the health vital data from at least one of a weighing scale, a wearable device, a food scale, an image acquisition device, a blood pressure meter, a gluco meter, an insulin injector and one or more cardiac monitoring tools. In some embodiment, receiving the health vital data from the one or more external user devices may include receiving at least one of bodyweight, blood pressure, glucose level, cholesterol level, pulse rate, amount of sleep, steps count, exercise-related data, weight and calorie intake of food, electrocardiogram data, haemoglobin level, oxygen level in blood and insulin from the one or more external user device. In a specific embodiment, receiving an information corresponding to oral or injectable medication of the user via an image acquisition device.

Furthermore, the method 300 includes organizing, by a data processing subsystem, received health vital data to create a medical data file representative of medical history of a user in step 320. The method 300 further includes analysing, by a medical data analysis subsystem, the processed medical data file using one or more analysis techniques in step 330. In one embodiment, analysing the medical data file using the one or more analysis techniques may include analysing the processed medical data file using at least one of a descriptive analysis technique, a predictive analysis technique, a prescriptive analysis technique and a comparative analysis technique.

Moreover, the method 300 includes generating, by the medical data analysis subsystem, one or more patterns corresponding to the health vital data based on analysed results in step 340. In one embodiment, the method may include receiving an access request from a third-party user to access the health vital data of the user. In such embodiment, the method 300 may include generate a permission grant request to access the health vital data by the third-party user. The method 300 further includes generating, by a recommendation subsystem, a report with one or more recommendations, wherein the one or more recommendations are provided by a healthcare provider based on the one or more patterns in step 350. In some embodiments, the method 300 may include suggesting one or more tools to the user corresponding to the one or more recommendations based on the one or more patterns.

In addition, the method 300 further includes generating, by a benchmark subsystem, a benchmark result based on the one or more patterns and the report with the one or more recommendations by the healthcare provider 360. The method 300 further includes providing, by the benchmark subsystem, the benchmark result of the user to one or more patients comprising at least one of a race, an age, a gender and weight corresponding to at least one of a race, an age, a gender and weight of the user based on permission of the user in step 370. In one embodiment, the method 300 may include receiving, by a notification subsystem, one or more reminders from the healthcare provider for the medicine dosages and preventive steps. In such embodiment, the method 300 may include generating one or more alerts for the user and kith and kin of the user, wherein the one or more alerts are corresponding to monitored data, the one or more recommendations and the one or more patterns.

In some embodiments, the method 300 may include receiving, by an insurer interface, a digital prescription from the healthcare provider, wherein the digital prescription is prescribed to an insured user. In such embodiment, the method 300 may include receive a health progress report of the insured user and the one or more tools used by the insured user. In a specific embodiment, the method 300 may include enabling, by a healthcare selection subsystem, the user to select a healthcare provider based on a location of the user.

In a preferred embodiment, the method 300 may include enabling, by a calibration subsystem, the user to remotely calibrate at least one of the one or more external user devices and the one or tools used by the user.

In some embodiments, the method 300 may include receiving, by a user experience sharing subsystem, a user experience corresponding to at least one of daily routine of diet, calories intake, exercise data, sleep patterns via at least one of a textual means, audio means and visual means.

Various embodiments of the health monitoring system for out-patient described above enables functioning of the system as a central repository of a patient's medical information. The system empowers patients to more easily manage their own healthcare decisions.

Furthermore, the inherent simplicity and low cost of the system makes it so attractive to healthcare providers and patients. Also, even in the case of the patient who only uses the health management device component of the system provides an invaluable window to the medical profession that enables proactive patient disease management thereby contributing greatly to the reduction in healthcare costs due to unforeseen complications that are widely known and attributed to disease.

Additionally, the remote aspect of this invention is a critical enhancement to automatic analysis, notification, and authorization. The system helps the patient to improve their chronic health dis-orders by obtaining vital data from various sensors, transducers and or portable meters and giving qualitative and quantitative vital data digitally about the patient's life style (Diet, Exercise, Stress) to healthcare providers to treat patients with correct medication, diet, exercise and stress related guidance, based on qualitative and quantitative vitals data provided through user health monitoring tools.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A health monitoring system for out-patient comprising:
a centralized platform located on a server and operatively coupled to one or more external user devices, wherein the centralized platform comprises:
a data receiving subsystem configured to receive health vital data from the one or more external user devices;
a data processing subsystem operatively coupled to the data receiving subsystem, wherein the data processing subsystem is configured to:
organize received health vital data to create a medical data file representative of medical history of a user;
a medical data analysis subsystem operatively coupled to the data processing subsystem, wherein the medical data analysis subsystem is configured to:
analyse the medical data file using one or more analysis techniques;
generate one or more patterns corresponding to the health vital data based on analysed results;
a recommendation subsystem operatively coupled to the analysis subsystem, wherein the recommendation subsystem is configured to generate a report with one or more recommendations, wherein the one or more recommendations are provided by a healthcare provider based on the one or more patterns;
a benchmark subsystem operatively coupled to the recommendation subsystem and medical data analysis subsystem, wherein the benchmark subsystem is configured to:
generate a benchmark result based on the one or more patterns and the report with one or more recommendations by the healthcare provider; and
provide the benchmark result of the user to one or more patients comprising at least one of a race, an age, a gender and weight corresponding to at least one of a race, an age, a gender and weight of the user based on permission of the user.

2. The system of claim 1, wherein the one or more external user devices comprises at least one of a weighing scale, a wearable device, a food scale, an image acquisition device, a blood pressure meter, a gluco meter, an insulin injector and one or more cardiac monitoring tools.

3. The system of claim 1, wherein the health vital data comprises at least one of bodyweight, blood pressure, glucose level, cholesterol level, pulse rate, amount of sleep, steps count, exercise-related data, weight and calorie intake of food, electrocardiogram data, haemoglobin level, oxygen level in blood, Saliva levels and insulin administered.

4. The system of claim 1, wherein the data receiving subsystem is configured to receive an information corresponding to oral or injectable medication of the user via an image acquisition device.

5. The system of claim 1, wherein the medical data analysis subsystem is configured to:
receive an access request from a third-party user to access the health vital data of the user; and
generate a permission grant request to access the health vital data by the third-party user.

6. The system of claim 1, wherein the recommendation subsystem is configured to suggest one or more tools to the user corresponding to the one or more recommendations based on the one or more patterns.

7. The system of claim 1, wherein the centralized platform comprises a notification subsystem receive one or more reminders from the healthcare provider for the medicine dosages and preventive steps.

8. The system of claim 7, wherein the notification subsystem is configured to generate one or more alerts to the user, kith and kin of the user, and to the healthcare provider, wherein the one or more alerts are corresponding to monitored data, the a report with one or more recommendations and the one or more patterns.

9. The system of claim 1, wherein the centralized platform comprises a healthcare selection subsystem configured to enable the user to select a healthcare provider based on a location of the user.

10. The system of claim 1, wherein the centralized platform comprises a calibration subsystem configured to enable the user to remotely calibrate at least one of the one or more external user devices and the one or tools used by the user.

11. The system of claim 1, wherein the centralized platform comprises a user experience sharing subsystem configured to receive a user experience corresponding to at least one of daily routine of diet, calories intake, exercise data, sleep patterns via at least one of a textual means, audio means and visual means.

12. The system of claim 1, further comprising an insurer interface operatively coupled to the centralized platform, wherein the insurer interface is configured to receive a digital prescription from the healthcare provider, wherein the digital prescription is prescribed to an insured user.

13. The system of claim 12, wherein the insurer interface is configured to receive a health progress report of the insured user and the one or more tools used by the insured user.

14. A method of health monitoring for an out-patient comprising:
receiving, by a data receiving subsystem, health vital data from the one or more external user devices;
organizing, by a data processing subsystem, received health vital data to create a medical data file representative of medical history of a user;
analysing, by a medical data analysis subsystem, the medical data file using one or more analysis techniques;
generating, by the medical data analysis subsystem, one or more patterns corresponding to the health vital data based on analysed results;
generating, by a recommendation subsystem, a report with one or more recommendations, wherein the one or more recommendations are provided by a healthcare provider based on the one or more patterns;
generating, by a benchmark subsystem, a benchmark result based on the one or more patterns and a report with the one or more recommendations by the healthcare provider; and
providing, by the benchmark subsystem, the benchmark result of the user to one or more patients comprising at least one of a race, an age, a gender and weight corresponding to at least one of a race, an age, a gender and weight of the user based on permission of the user.

15. The method of claim 14, further comprising receiving, by a notification subsystem, one or more reminders from the healthcare provider for the medicine dosages and preventive steps.

16. The method of claim 14, further comprising:
receiving, by an insurer interface, a digital prescription from the healthcare provider, wherein the digital prescription is prescribed to an insured user; and
receiving, by the insurer interface, a health progress report of the insured user and the one or more tools used by the insured user.

17. The method of claim 14, further comprising receiving, by a rating subsystem, a rating from the user corresponding to medication recommendation, the one or more tools recommendation and monitoring service provided by the healthcare provider.

18. The method of claim 14, further comprising receiving, by a user experience sharing subsystem, a user experience corresponding to at least one of daily routine of diet, calories intake, exercise data, sleep patterns via at least one of a textual means, audio means and visual means.
